(19) United States
(12) Patent Application Publication
(10) Pub. No.: US 2025/0381388 A1
(43) Pub. Date: Dec. 18, 2025

(54) ANTEGRADE PERFUSION ASSEMBLY

(71) Applicant: Narula Health Care Consultants, LLC, Orwigsburg, PA (US)

(72) Inventor: Arvin Narula, La Jolla, CA (US)

(21) Appl. No.: 19/236,108

(22) Filed: Jun. 12, 2025

Related U.S. Application Data
(60) Provisional application No. 63/658,982, filed on Jun. 12, 2024.

(51) Int. Cl. A61M 60/851; A61M 1/36; A61M 25/00; A61M 39/10; A61M 39/22; A61M 60/117; A61M 60/34; A61M 60/38

(52) U.S. Cl. CPC A61M 60/851; A61M 1/3627; A61M 39/22; A61M 60/117; A61M 60/34; A61M 60/38; A61M 25/005; A61M 2039/1088; A61M 2039/229

(57) ABSTRACT

An antegrade perfusion assembly may have a braided sheath, a three-way stopcock, and a male-to-male connection. The three-way stopcock can fluidly communicate with the braided sheath in assembly. The three-way stopcock can furnish control of perfusate flow amid use of the antegrade perfusion assembly, and can facilitate de-airing of the braided sheath amid use of the antegrade perfusion assembly. The male-to-male connection can fluidly communicate with the three-way stopcock, and can accept reception and securement with an oxygenated blood source amid use of the antegrade perfusion assembly.

### CROSS-REFERENCE TO RELATED APPLICATION

This application claims the benefit of U.S. Provisional Patent Application No. 63/658,982, filed Jun. 12, 2024.

### TECHNICAL FIELD

This disclosure relates generally to medical devices and assemblies employed in surgery on human patients and, more particularly, to antegrade perfusion procedures and systems.

### BACKGROUND

Cardiogenic shock is a life-threatening condition characterized by the heart's inability to pump blood effectively, resulting in inadequate cardiac output and blood pressure. Patients in cardiogenic shock often require life-stabilizing medications to maintain their blood pressure and sustain vital organ function. To address this condition, various mechanical circulatory support devices have been developed to provide temporary cardiac support when conventional treatments are insufficient. But the use of these devices, particularly those requiring large bore access, poses a significant challenge—the risk of limb ischemia.

Large bore access is typically necessary for the effective deployment of mechanical circulatory support devices in cardiogenic shock patients. These devices maintain perfusion to vital organs when the heart is unable to do so. Unfortunately, these devices can sometimes inadvertently obstruct blood flow through the affected artery, jeopardizing the patient's limbs and overall well-being. Limb ischemia, if left untreated, can lead to severe complications, including amputation, even if the patient initially survives the cardiac arrest episode.

One existing approach to mitigate the risk of limb ischemia in these situations is to perfuse the affected limb with a catheter. But the current standard catheters used for antegrade perfusion have inherent limitations that can compromise their effectiveness. These limitations can result in clot formation within the antegrade perfusion system, leading to suboptimal limb perfusion and, in some cases, premature discontinuation of the mechanical circulatory support device. Such complications can have unwanted consequences.

A factor contributing to flow limitations in the antegrade perfusion system is the presence of stopcocks. These stopcocks serve as valve mechanisms within the system, controlling the flow of perfusate to the limb. Unfortunately, these stopcocks can become limiting reagents, impeding the flow of blood and perfusate through the system. This restriction not only affects limb perfusion but also increases the risk of clot formation and device malfunction.

### SUMMARY

In an embodiment, an antegrade perfusion assembly may include a braided sheath, a three-way stopcock, and a male-to-male connection. The three-way stopcock is fluidly communicated with the braided sheath amidst assemblage of the antegrade perfusion assembly. The three-way stopcock serves to furnish control of perfusate flow during use of the antegrade perfusion assembly. The three-way stopcock further serves to facilitate de-airing of the braided sheath during use of the antegrade perfusion assembly. The male-to-male connection is fluidly communicated with the three-way stopcock amidst assemblage of the antegrade perfusion assembly. During use of the antegrade perfusion assembly, the male-to-male connection can accept securement of an oxygenated blood source.

In an embodiment, an antegrade perfusion assembly may include a braided sheath, a three-way stopcock, and a male-to-male connection. The braided sheath has a metal braided portion and a non-metal tube portion. The braided portion and non-metal tube portion are in fluid communication with each other. The three-way stopcock is fluidly communicated with the braided sheath amidst assemblage of the antegrade perfusion assembly. The three-way stopcock serves to furnish control of perfusate flow during use of the antegrade perfusion assembly. The three-way stopcock further serves to facilitate de-airing of the braided sheath during use of the antegrade perfusion assembly. The three-way stopcock has a first diametrically-enlarged portion and a second diametrically-enlarged portion. The second diametrically-enlarged portion is located at an opposite side of the three-way stopcock with respect to the first diametrically-enlarged portion. The male-to-male connection is fluidly communicated with the three-way stopcock amidst assemblage of the antegrade perfusion assembly. During use of the antegrade perfusion assembly, the male-to-male connection can accept securement of an oxygenated blood source.

Further scope of applicability of the present disclosure will become apparent from the detailed description given hereinafter. But it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF DRAWINGS

The present disclosure will become more fully understood from the detailed description given below and the accompanying drawings, which are given by way of illustration only, and do not limit the present disclosure, and wherein:

FIG. 1 is a schematic depiction of an embodiment of a three-way stopcock;

FIG. 2 is a schematic depiction of another embodiment of a three-way stopcock; and

FIG. 3 is a schematic depiction of an embodiment of an antegrade perfusion assembly having embodiments of a braided sheath, the three-way stopcock of FIG. 2, and a male-to-male connection.

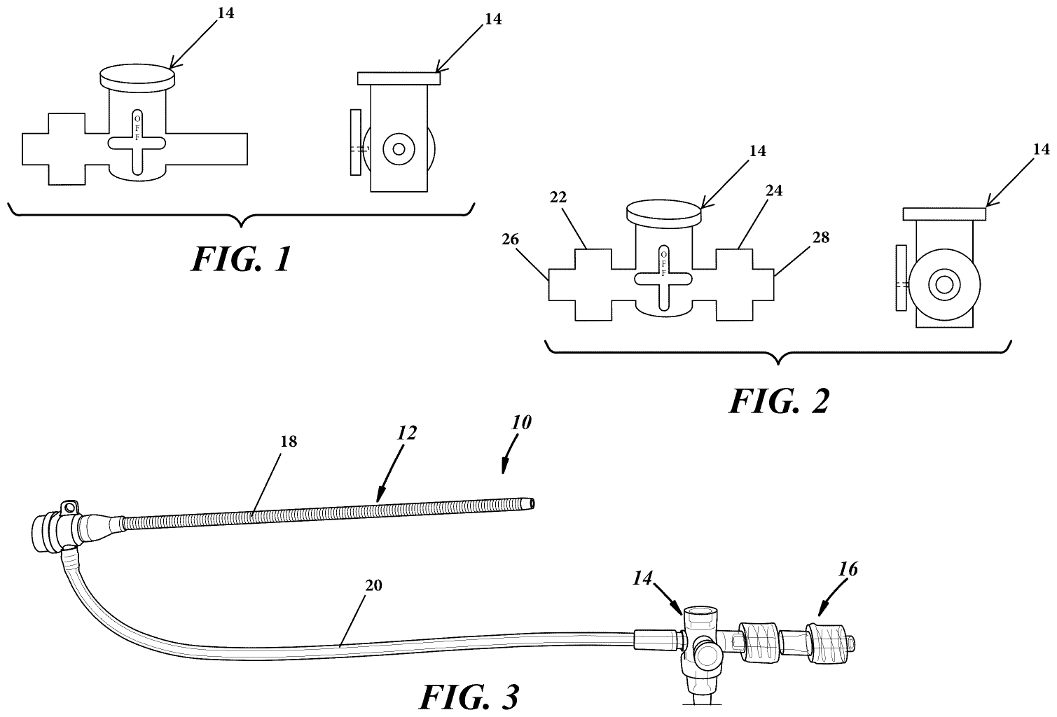

### DETAILED DESCRIPTION

The figures present an embodiment of an antegrade perfusion assembly 10 that is suitable for use in a medical procedure on a human patient. The antegrade perfusion assembly 10 can exhibit various designs, constructions, and components in various embodiments. In the embodiment of FIG. 3—and unlike heretofore known devices—the antegrade perfusion assembly 10 includes a braided sheath 12, a three-way stopcock 14, and a male-to-male connection 16; still, other embodiments of the antegrade perfusion assembly could have more, less, and/or different components, and the individual components themselves could exhibit various designs, constructions, and components than those presented herein. In the embodiment of FIG. 3, the braided sheath 12 fluidly communicates with the three-way stopcock 14, which in turn fluidly communicates with the male-to-male connection 16. Fluid-flow can hence travel among the braided sheath 12, three-way stopcock 14, and male-to-male connection 16 during use of the antegrade perfusion assembly 10. The braided sheath 12 has direct and immediate connection with the three-way stopcock 14, and the three-way stopcock 14 has direct and immediate connection with the male-to-male connection 16. The three-way stopcock 14 is interposed and situated in-between the braided sheath 12 and male-to-male connection 16.

To address the challenges of known approaches, and thereby improve limb perfusion in cardiogenic shock patients requiring large bore access and mechanical circulatory support, the proposed solution is to develop an all-in-one kit specifically designed for antegrade perfusion: the antegrade perfusion assembly 10. The antegrade perfusion assembly 10 aims to enhance the flow of perfusate to the affected limb, reduce the risk of clot formation, and provide a more efficient, effective, and reliable means of limb perfusion. By eliminating the limitations associated with current catheters and stopcocks of the past approaches, the antegrade perfusion assembly 10 has been found to improve patient outcomes, decrease the risk of limb complications, and ultimately save lives in the context of cardiogenic shock and mechanical circulatory support.

The embodiments described herein relate to an innovative antegrade perfusion system designed to address the limitations associated with current catheter-based limb perfusion methods in patients with cardiogenic shock. The antegrade perfusion assembly 10 offers a unique combination of features, including the braided sheath 12 to prevent kinking, the three-way stopcock 14 for de-airing, and the male-to-male connection 16 for oxygenated blood perfusion.

Braided Sheath 12. The antegrade perfusion assembly 10 includes the braided sheath 12 that encompasses the perfusion catheter. The braided sheath 12 is designed to significantly reduce the risk of kinking or collapsing of the catheter during use. The braided structure of the braided sheath 12 imparts overall flexibility and strength to the braided sheath 12, ensuring that it maintains its shape and allows for smooth and uninterrupted blood flow to the limb. The braided sheath 12, per the embodiment of FIG. 3, has a braided portion 18 and a tube portion 20. The tube portion 20 extends from the braided portion 18 and fluidly communicates therewith. The braided portion 18 can be composed of a metal braided structure, and the tube portion 20 can be composed of a non-metal material such as a plastic material.

Three-Way Stopcock 14. Incorporated into the antegrade perfusion assembly 10 is the three-way stopcock 14. The three-way stopcock 14 serves multiple functions within the antegrade perfusion assembly 10, per this embodiment. Firstly, the three-way stopcock 14 allows for the precise control of perfusate flow, ensuring that blood flow to the limb can be adjusted as needed. Secondly, the three-way stopcock 14 facilitates the de-airing of the braided sheath 12, preventing air embolisms and ensuring the safety of the patient. This feature enhances the reliability and safety of the perfusion process when employing the antegrade perfusion assembly 10 in use. In the embodiment of FIGS. 2 and 3, the three-way stopcock 14 has a first diametrically-enlarged portion 22 and a second diametrically-enlarged portion 24. The first diametrically-enlarged portion 22 is located adjacent and distanced from a first open end 26 of the three-way stopcock 14. The first open end 26 accepts connection with the braided sheath 12. The second diametrically-enlarged portion 24, on the other hand, is located adjacent and distanced from a second open end 28 of the three-way stopcock 14. The second open end 28 accepts connection with the male-to-male connection 16. The first diametrically-enlarged portion 22 is located on an opposite side of the three-way stopcock 14 with respect to the second diametrically-enlarged portion 24.

Male-to-Male Connection 16. At the back end of the three-way stopcock 14, the male-to-male connection 16 is secured. The male-to-male connection 16 is designed and constructed to seamlessly attach to a perfusion tube carrying oxygenated blood. The male-to-male connection 16 ensures a secure and leak-free interface between the antegrade perfusion assembly 10 and the source of oxygenated blood, which may be provided by an external perfusion device or other oxygenation equipment.

The antegrade perfusion assembly 10, per at least some embodiments, may provide several advantages over existing methods and devices:

Improved Safety. The inclusion of the three-way stopcock 14 for de-airing minimizes the risk of air embolisms, a safety concern in perfusion procedures, per at least one embodiment of the antegrade perfusion assembly 10.

Enhanced Flow Control. The three-way stopcock 14 allows for precise control over perfusate flow rates, ensuring optimal limb perfusion while minimizing the risk of complications, per at least one embodiment of the antegrade perfusion assembly 10.

Reduced Risk of Kinking. The braided sheath's 12 design greatly reduces the risk of kinking or collapsing of the catheter, ensuring continuous and unobstructed blood flow, per at least one embodiment of the antegrade perfusion assembly 10.

Reliable Connection. The male-to-male connection 16 at the back end of the three-way stopcock 14 provides a secure and leak-free interface for perfusion with oxygenated blood, per at least one embodiment of the antegrade perfusion assembly 10.

Moreover, per at least one embodiment, the antegrade perfusion assembly 10 can exhibit a kit-like and all-in-one configuration in which the components are somewhat permanently connection with one another. That is, the braided sheath 12 can have a non-disconnectable attachment to the three-way stopcock 14, and the three-way stopcock 14 can in turn have a non-disconnectable attachment to the male-to-male connection 16. This configuration is thought to facilitate and enhance overall usage and effectiveness of the antegrade perfusion assembly 10.

In summary, the antegrade perfusion assembly 10 provides an advancement in the field of limb perfusion for cardiogenic shock patients. Its combination of features, per at least some embodiments, addresses key drawbacks of existing systems, providing improved safety, flow control, and reliability in limb perfusion procedures.

In general, while a multitude of embodiments have been depicted and described with a multitude of components in each embodiment, in alternative embodiments of the antegrade perfusion assembly the components of various embodiments could be intermixed, combined, and/or exchanged for one another. In other words, components described in connection with a particular embodiment are not necessarily exclusive to that particular embodiment.

As used herein, the terms “general,” “generally,” “approximately,” and “substantially” are intended to account for the inherent degree of variance and imprecision that is often attributed to, and often accompanies, any design and manufacturing process and measurement, including engineering tolerances, and without deviation from the relevant functionality and intended outcome, such that mathematical precision and exactitude is not implied and, in some instances, is not strictly possible. In other instances, the terms “general,” “generally,” “approximately,” and “substantially” are intended to represent the inherent degree of uncertainty that is often attributed to any quantitative comparison, value, and measurement calculation, or other representation, such that mathematical precision and exactitude is not implied and, in some instances, is not strictly possible.

It is to be understood that the foregoing description is not a definition of the invention, but is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” and “such as,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

Those of skill in the art will understand that modifications (additions and/or removals) of various components of the substances, formulations, apparatuses, methods, systems, and embodiments described herein may be made without departing from the full scope and spirit of the present disclosure, which encompass such modifications and any and all equivalents thereof.

1. An antegrade perfusion assembly, the assembly comprising:
a braided sheath;
a three-way stopcock fluidly communicable with said braided sheath, said three-way stopcock furnishing control of perfusate flow during use of the antegrade perfusion assembly and facilitating de-airing of said braided sheath during use of the antegrade perfusion assembly; and
a male-to-male connection fluidly communicable with said three-way stopcock, said male-to-male connection accepting securement of an oxygenated blood source during use of the antegrade perfusion assembly.

2. The antegrade perfusion assembly as set forth in claim 1, wherein said braided sheath has direct connection with said three-way stopcock, and said three-way stopcock has direct connection with said male-to-mail connection, said three-way stopcock interposed between said braided sheath and said male-to-male connection.

3. The antegrade perfusion assembly as set forth in claim 1, wherein said braided sheath has a metal braided portion and a non-metal tube portion in fluid communication with each other.

4. The antegrade perfusion assembly as set forth in claim 1, wherein said three-way stopcock has a first diametrically-enlarged portion and a second diametrically-enlarged portion located at an opposite side of said three-way stopcock with respect to said first diametrically-enlarged portion.

5. The antegrade perfusion assembly as set forth in claim 1, wherein said braided sheath has a non-disconnectable attachment to said three-way stopcock, and said three-way stopcock has a non-disconnectable attachment to said male-to-male connection.

6. An antegrade perfusion assembly, the assembly comprising:
a braided sheath, said braided sheath having a metal braided portion and a non-metal tube portion in fluid communication with each other;
a three-way stopcock fluidly communicable with said braided sheath, said three-way stopcock furnishing control of perfusate flow during use of the antegrade perfusion assembly and facilitating de-airing of said braided sheath during use of the antegrade perfusion assembly, said three-way stopcock having a first diametrically-enlarged portion and a second diametrically-enlarged portion located at an opposite side of said three-way stopcock with respect to said first diametrically-enlarged portion; and
a male-to-male connection fluidly communicable with said three-way stopcock, said male-to-male connection accepting securement of an oxygenated blood source during use of the antegrade perfusion assembly.

7. The antegrade perfusion assembly as set forth in claim 6, wherein said braided sheath has direct connection with said three-way stopcock, and said three-way stopcock has direct connection with said male-to-mail connection, said three-way stopcock interposed between said braided sheath and said male-to-male connection.
